(19) United States
(12) Patent Application Publication
(10) Pub. No.: US 2025/0091975 A1
(43) Pub. Date: Mar. 20, 2025

(54) PREPARATION METHOD FOR SGLT2 INHIBITOR INTERMEDIATE V

(71) Applicant: WANBANG BIOPHARMACEUTICALS, Xuzhou, Jiangsu Province (CN)

(72) Inventors: Keyan WANG, Xuzhou (CN); Shibin WU, Xuzhou (CN); Ruiyan HAN, Xuzhou (CN); Xinmeng ZHU, Xuzhou (CN); Chunlei LI, Xuzhou (CN); Zeng WU, Xuzhou (CN)

(73) Assignee: WANBANG BIOPHARMACEUTICALS, Xuzhou, Jiangsu Province (CN)

(21) Appl. No.: 18/730,648

(22) PCT Filed: Mar. 16, 2022
(86) PCT No.: PCT/CN2022/081072

§ 371 Date: Jul. 19, 2024

(30) Foreign Application Priority Data
Jan. 26, 2022 (CN) 202210092840.X

(51) Int. Cl. C07C 41/22; C07C 41/48; C07D 407/14; C07F 5/02; C07F 7/18

(52) U.S. Cl. CPC C07C 41/22; C07C 41/48; C07D 407/14; C07F 5/022; C07F 7/1892; C07C 2531/02

(57) ABSTRACT

Disclosed is a preparation method for an SGLT2 inhibitor intermediate V, the method comprising: adding a compound IV to a solvent, and in the presence of a catalytic reagent, carrying out heat preservation reaction with a fluorination reagent. The preparation process for the intermediate V is simple in operations, mild in reaction conditions and high in safety, facilitates quality control and is suitable for industrial production. In addition, the intermediate V prepared by the method is high in purity, has few side reaction product monofluoro-impurity shown as structural Formula V′, is easy to purify, and is applicable to quality research on active pharmaceutical ingredients and preparations of the SGLT2 inhibitor.

### TECHNICAL FIELD

The present invention relates to the technical field of medicinal chemistry, and more specifically to a preparation method for a sodium-dependent glucose transporter 2 (SGLT2) inhibitor intermediate V.

### BACKGROUND

Formula VI represents a family of aryl, heteroaryl, O-aryl and O-heteroaryl carbasugar compounds, which function as a sodium-dependent glucose transporter 2 (SGLT2) inhibitor.

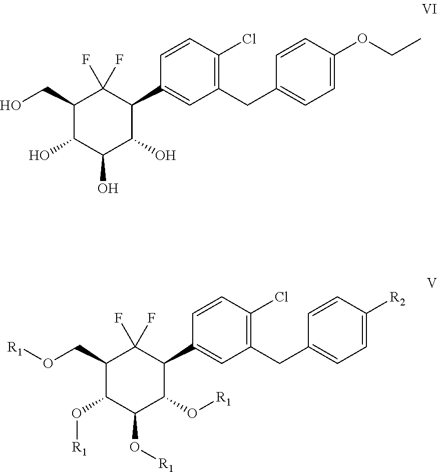

Intermediate V is an important intermediate in the synthesis of SGLT2 inhibitor VI. In the synthesis method described in the example of CN104909997B, the yield of the intermediate in the fluorination process of carbonyl is not so good, and the conversion rate is even less desirable when the carbonyl group at a position with large steric hindrance is fluorinated. According to the inventor's research, the main reason is that a monofluoro-impurity shown as structural Formula V′ is easily produced in the fluorination process of carbonyl, and the content is high (about 28-46%).

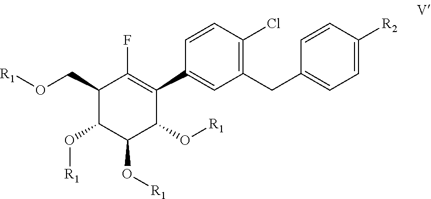

To ensure the safety of the SGLT2 inhibitor and effectively control its quality, it is of great significance to further study the fluorination process of carbonyl.

### SUMMARY

To overcome the shortcomings existing in the prior art, the present invention aims to provide a safe, simple and high-efficiency preparation method for an SGLT2 inhibitor intermediate V. On the basis of fully studying the synthesis process, efficient preparation conditions for fluorination of carbonyl in the method of the present invention are developed. The method of the present invention obviously reduces the impurity content, reduces the number of purification processes, improves production safety and yield, and is suitable for industrial production.

To achieve the above object, the following technical solutions are adopted in the present invention.

The intermediate V of the present invention is an SGLT2 inhibitor intermediate, having a structure of Formula (I):

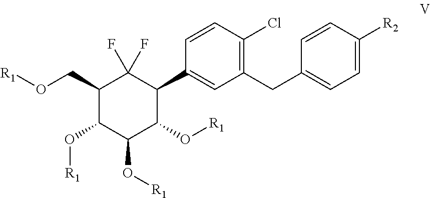

where: R₁ is benzyl, p-methoxybenzyl, triphenylmethyl, acetyl, benzoyl, pivaloyl, trimethylsilyl, t-butyldimethylsilyl, t-butyldiphenylsilyl, tri-iso-propylsilyl, 2-tetrahydropyranyl, methoxymethyl or 2-ethoxyethyl, and R₂ is methyl, ethyl, methoxy, ethoxy, or (tetrahydrofuran-3-yl)oxy.

In a specific embodiment, R1 is benzyl, p-methoxybenzyl, trimethylsilyl, 2-tetrahydropyranyl or methoxymethyl; and R2 is ethoxy.

The present invention aims to provide a preparation method for an SGLT2 inhibitor intermediate V, which includes subjecting a compound of Formula IV to a substitution reaction with a fluorination reagent in the presence of a catalytic reagent, where R₁, and R₂ are as defined above. The synthesis route is as follows:

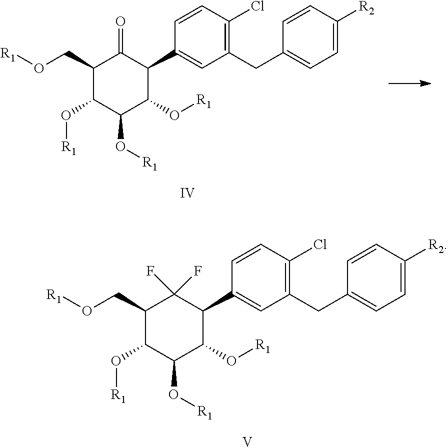

The raw material IV used in the present invention can be obtained according to the synthetic method described in the example of CN104909997B or a combination thereof with a generally known method.

In some embodiments, the preparation method of the present invention specifically includes: adding the compound of Formula IV to a solvent, and in the presence of a catalytic reagent, carrying out heat-preservation substitution reaction with a fluorination reagent, to obtain the SGLT2 inhibitor intermediate V.

In some embodiments, the catalytic reagent mentioned in the present invention is one or more of methanol, anhydrous ethanol, isopropanol, n-propanol or n-butanol. In a preferred embodiment of the present invention, the catalytic reagent in the step is preferably one of anhydrous ethanol, methanol and n-propanol, and more preferably anhydrous ethanol.

In some embodiments, the amount of the catalytic reagent mentioned in the present invention is 2-6 wt ‰ of the amount of the compound of Formula IV. In a preferred embodiment of the present invention, the amount of the catalytic reagent in the step is 2 wt ‰ of the amount of the compound of Formula IV.

By adding the catalytic reagent in the present invention, the production of the impurity of Formula V′ is significantly reduced, and the yield of the compound of Formula V is significantly improved.

In some embodiments, the solvent mentioned in the present invention is one or more selected from dichloromethane, acetonitrile, isopropyl ether, tetrahydrofuran, methyl tert-butyl ether, n-heptane and ethyl acetate. In a preferred embodiment of the present invention, the solvent in the step is one of dichloromethane, acetonitrile, isopropyl ether and tetrahydrofuran, and more preferably dichloromethane.

In some embodiments, the amount of the solvent mentioned in the present invention is (0-10) ml/g relative to the weight of the compound of Formula IV. In a preferred embodiment of the present invention, the amount of the solvent used in step is (3-6) ml/g, and more preferably 5 ml/g, relative to the weight of the compound of Formula IV.

In some embodiments, the fluorination reagent of the present invention is one selected from diethylaminosulfur trifluoride, 1-fluoropyridinium tetrafluoroborate, anhydrous hydrogen fluoride, anhydrous potassium fluoride, DAST fluoroborate, selective fluorination reagent II (CAS No. 159269-48-4), bis(2-methoxyethyl)aminosulfur trifluoride, and hydrogen fluoride pyridine complex. In a preferred embodiment of the present invention, the fluorination reagent in the step is one of diethylaminosulfur trifluoride, DAST fluoroborate, selective fluorination reagent II, and bis(2-methoxyethyl)aminosulfur trifluoride, and preferably diethylaminosulfur trifluoride.

In some embodiments, the molar amount of the fluorination reagent mentioned in the present invention is 2-30 times the molar amount of the compound of Formula IV. In some specific embodiments, the molar amount of the fluorination reagent is 2-15 times the molar amount of the compound of Formula IV In a specific embodiment of the present invention, when the fluorination reagent in the step is diethylaminosulfur trifluoride, the molar amount is 15 times the molar amount of the compound of Formula IV.

In some embodiments, the temperature of the heat-preservation substitution reaction in the present invention is 10-70° C. In a preferred embodiment of the present invention, the temperature of the heat-preservation substitution reaction is 18-25° C. or 19-25° C.

In some embodiments, the reaction time for the heat-preservation substitution reaction in the present invention is 10-98 hrs. In a preferred embodiment of the present invention, the reaction time for the heat-preservation substitution reaction in the step is 50-80 hrs, or further 65-72 hrs.

The present invention further provides use of the SGLT2 inhibitor intermediate V in the preparation of an SGLT2 inhibitor VI. In the present invention, the SGLT2 inhibitor VI can be prepared by deprotecting the compound of Formula V, for example, according to the method described in the example in CN104909997B, to obtain the SGLT2 inhibitor VI.

Compared with the prior art, the present invention has the following beneficial effects:

The present invention provides a preparation method for an SGLT2 inhibitor intermediate V. The preparation process is simple operation, mild in reaction conditions, and high in safety, facilitates quality control and is suitable for industrial production. In addition, the crude product of the SGLT2 inhibitor intermediate V prepared by the method is high in purity, has a significantly lowered content (2.5‰ r less) of the side reaction product monofluoro-impurity shown as structural Formula V′, is easy to purify, has high yield, and is applicable to the quality research on active pharmaceutical ingredients and preparation of the SGLT2 inhibitor.

### BRIEF DESCRIPTION OF THE DRAWINGS

FIG. 1 is a ¹H NMR spectrum of a monofluoro-impurity V′;

FIG. 2 is a ¹³C NMR spectrum of the monofluoro-impurity V′;

FIG. 3 is an IR spectrum of the monofluoro-impurity V′;

FIG. 4 is an UV spectrum of the monofluoro-impurity V′;

FIG. 5 is a LC-MS spectrum of the monofluoro-impurity V′; and

FIG. 6 is an HPLC chromatogram of the monofluoro-impurity V.

### DETAILED DESCRIPTION

Specific embodiments of the present invention are provided below to show possible implementation processes, but not to limit the present invention. To make the objects, the technical solution, and advantages of the present invention clearer, the present invention is described in further detail with reference to examples. It should be understood that the specific embodiments described herein are merely used to explain the present invention but are not intended to limit the present invention.

Unless otherwise specified, the monofluoro-impurity mentioned in the following examples is a monofluoro-impurity of Formula V′ below:

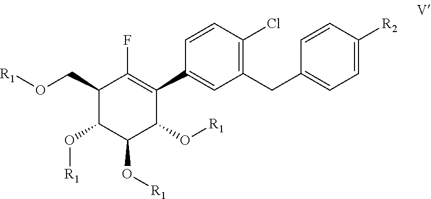

where R1 and R2 have the same definitions as those for the compound V in specific embodiments.

### Example 1

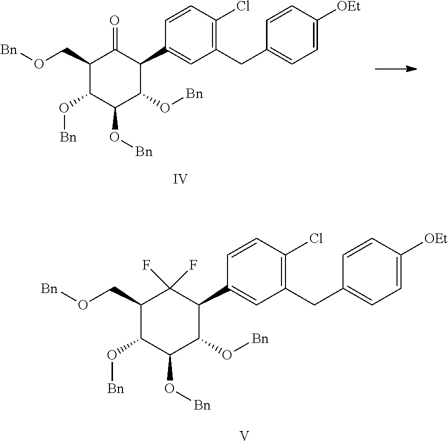

(2R,3R,4R,5S,6S)-3,4,5-tri(benzyloxy)-2-((benzyloxy)methyl)-6-(4-chloro-3-(4-ethoxyb enzyl)phenyl)cyclohexanone (5.0 g, 6.4 mmol, 1.0 eq), dichloromethane (25 mL), diethylaminosulfur trifluoride (15.4 g, 95.5 mmol, 15.0 eq), and anhydrous ethanol (0.01 g, 2‰ M_{SM}) were added to a reaction flask, heated to 20° C. and reacted for 72 hrs. The reaction solution was quenched with iced water, and alkalized. The organic phase was separated, dried, and concentrated to obtain a crude product of Formula V (5.04 g, purity 93.2%, monofluoro-impurity 1.3%, yield 98.0%).

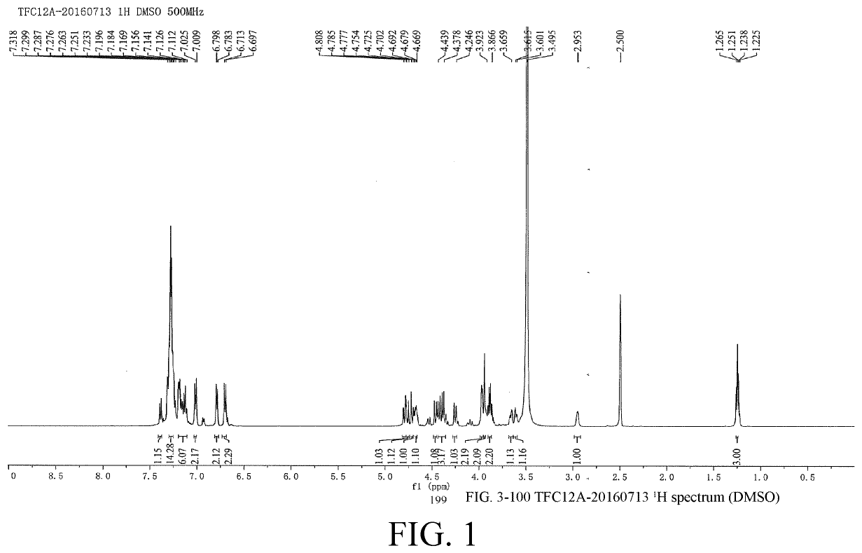

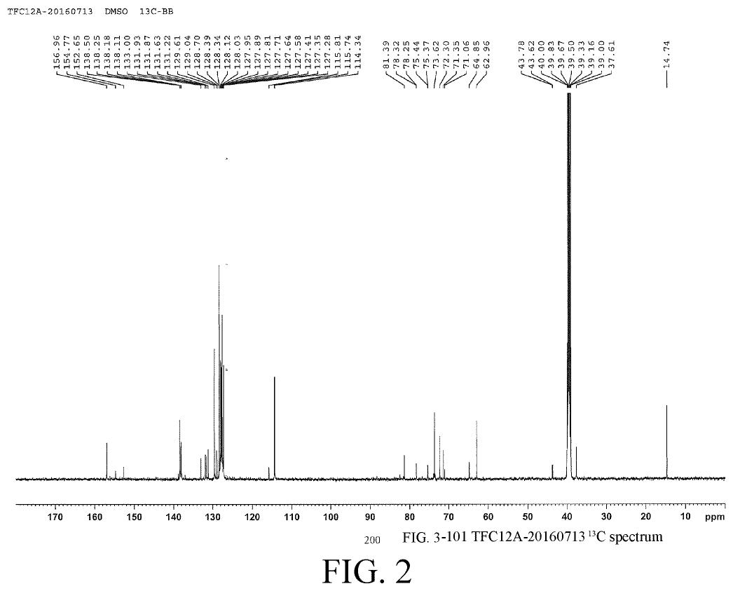

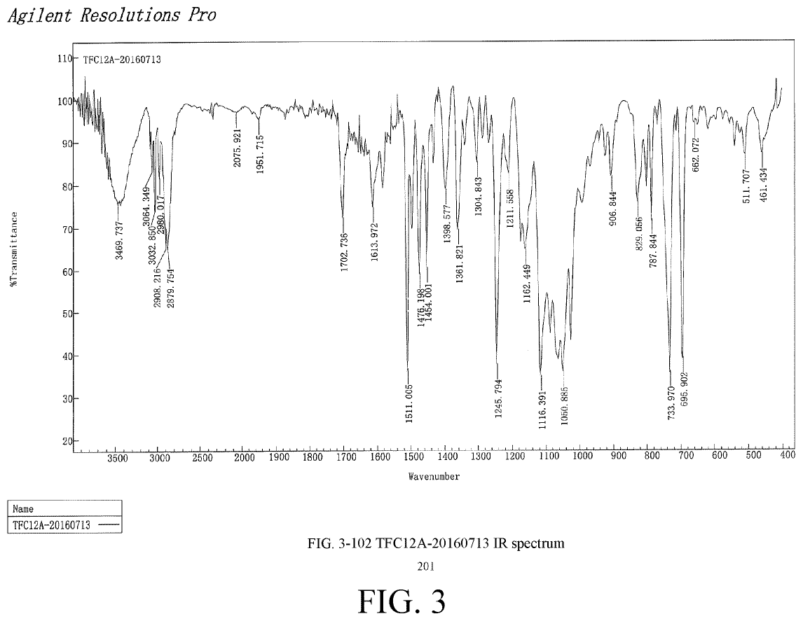

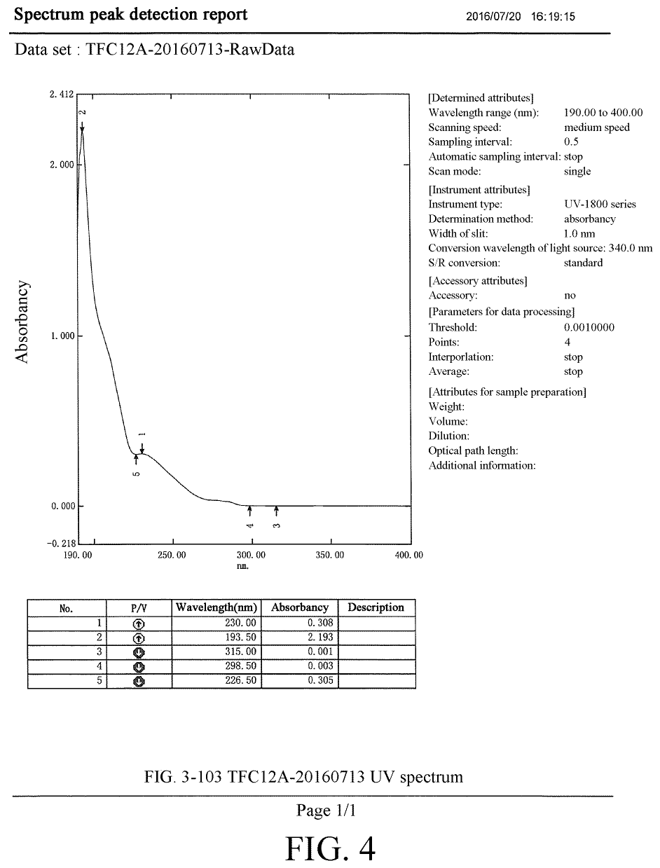

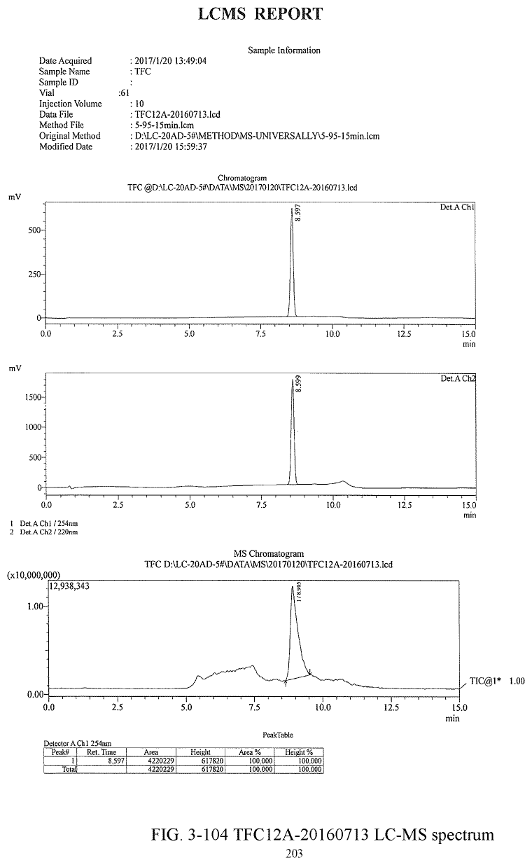

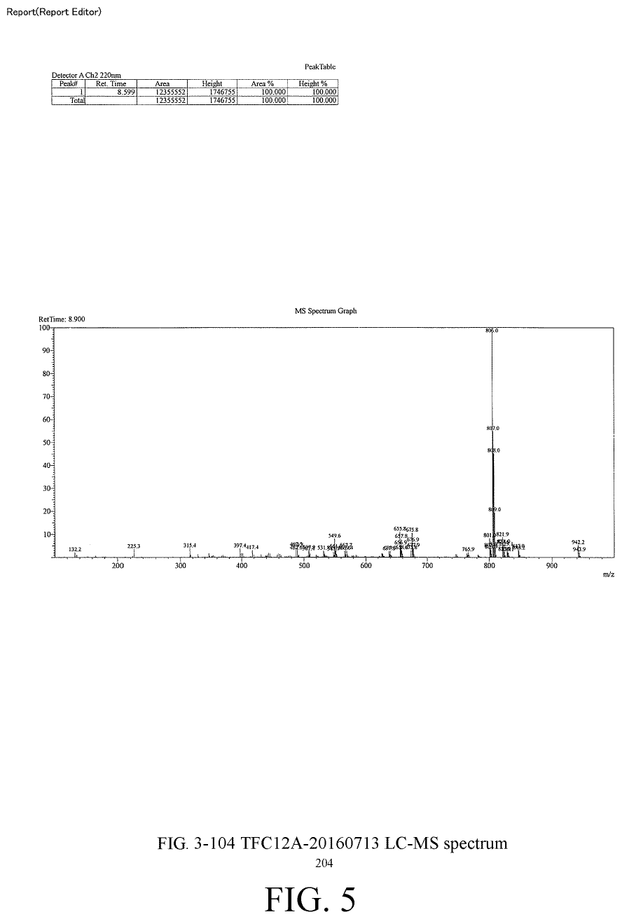

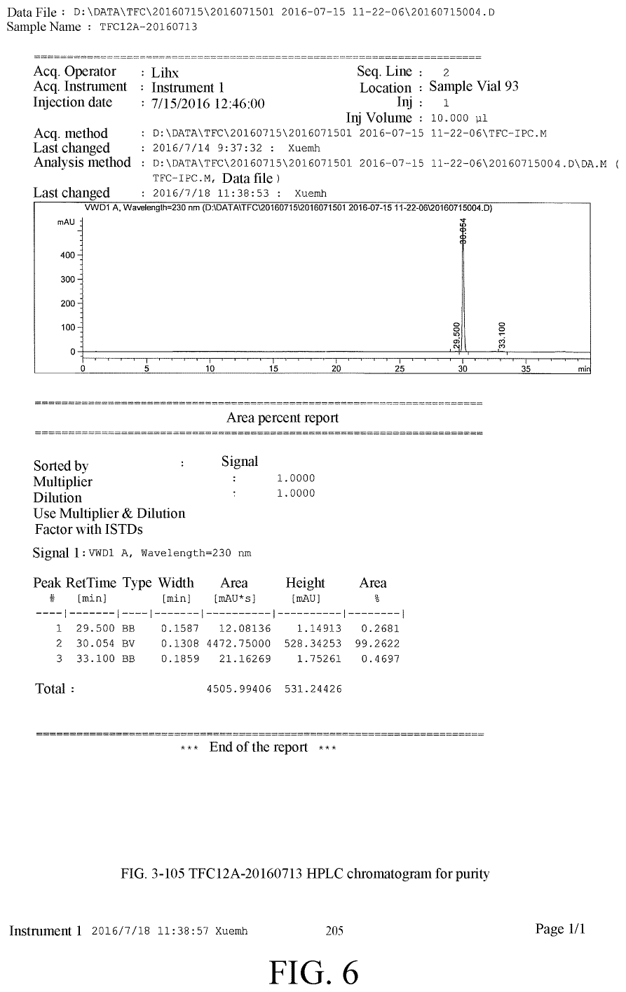

The structure characterization data of the monofluoro-impurity V is shown in FIGS. 1-6 and Table 1.

TABLE 1
Structure characterization data of monofluoro-impurity V′

| Item | Feature data | Feature attribution |
|---|---|---|
| ¹H NMR | 7.381-7.397 (d, 1H, J = 8 Hz), 7.233-7.299 (m, | Phenyl ring proton |
| (D6-DMSO, | 14H), 7.112-7.184 (m, 6H), 7.009-7.025 (d, 2H, | |
| δ ppm), | J = 12 Hz), 6.783-6.798(d, 2H, J = 7.5 Hz), | |
| 500 MHz | 6.697-6.713 (d, 2H, J = 8 Hz) | |
| | 4669-4.808 (m, 4H), 4354-4478 (m, 4H), | / |
| | 4.246-4.268 (d, 1H, J = 11 Hz), 3.975 (m, 2H), | |
| | 3.946 (s, 2H), 3.659 (m, 1H), 3.615 (m, 1H), | |
| | 2.953 (m, 1H) | |
| | 3.866-3.908 (q, 2H), 1.251(t, 3H, J = 7 Hz) | Ethyl proton |
| ¹³C NMR | 156.96, 138.50, 138.25, 138.18, 138.11, 133.00, | Carbon in unsaturated |
| (D6-DMSO, | 131.93, 131.87, 131.63, 131.22, 129.61, 129.04, | region |
| δ ppm), | 128.70, 128.39, 128.34, 128.12, 128.03, 127.95, | |
| 125 MHz | 127.89, 127.81, 127.71, 127.64, 127.58, 127.41, | |
| | 127.35, 127.28, 114.34 | |
| | 81.39, 78.32, 78.25, 75.44, 75.37, 73.62, 72.30, | Carbon in partially |
| | 71.35, 71.06, 64.85 | saturated region |
| | 62.96, 14.74 | Ethyl carbon of ethoxy |
| | 43.78, 43.62 | Allylic methylene |
| | | carbon |
| | 37.61 | Benzyl carbon of |
| | | ethoxybenzyl |
| LCMS | m/z = 806 | M + Na⁺ |
| IR | 3064.349, 3032.850 | C—H stretching |
| | | vibration of benzene |
| | | ring |
| | 2980.017, 2908.216, 2879.754 | Methyl/methylene |
| | | stretching vibration |
| | 1613.972, 1511.005, 1476.198 | Benzene ring skeleton |
| | | vibration |
| | 1245.794, 1162.449, 1116.391 | Stretching vibration in |
| | | ether |
| | 733.970, 695.902 | C—H bending vibration |
| | | of benzene ring |
| UV | 230.00 | K band |
| | 193.50 | E band |

### Example 2

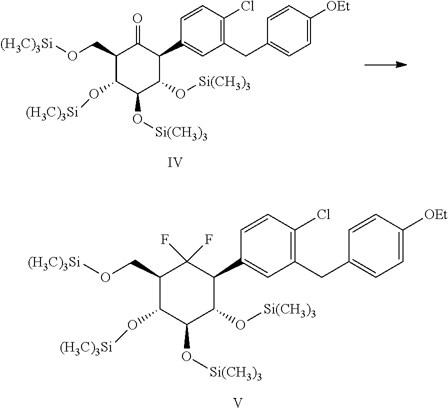

(2R,3R,4R,5S,6S)-3,4,5-tris(trimethylsilyloxy)-2-((trimethylsilyloxy)methyl)-6-(4-chloro-3-(4-ethoxybenzyl)phenyl)cyclohexanone (5.0 g, 7.0 mmol, 1.0 eq), acetonitrile (18 mL), diethylaminosulfur trifluoride (7.4 g, 45.9 mmol, 6.56 eq), and anhydrous ethanol (0.03 g, 6‰ M_{SM}) were added to a reaction flask, heated to 23° C. and reacted for 68 hrs. The reaction solution was quenched with iced water, and alkalized. The organic phase was separated, dried, and concentrated to obtain a crude product of Formula V (4.98 g, purity 91.6%, monofluoro-impurity 2.1%, yield 97.1%).

### Example 3

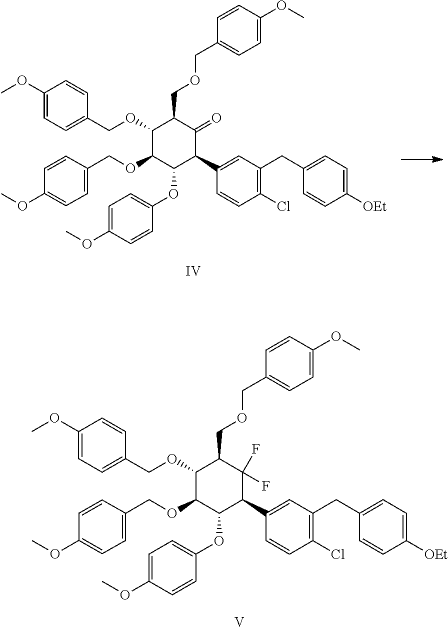

(2R,3R,4R,5S,6S)-3,4,5-tris(p-methoxybenzyloxy)-2-((p-methoxybenzyloxy)methyl)-6-(4-chloro-3-(4-ethoxybenzyl)phenyl)cyclohexanone (5.0 g, 5.6 mmol, 1.0 eq), isopropyl ether (20 mL), DAST fluoroborate (10.5 g, 45.8 mmol, 8.18 eq), and methanol (0.015 g, 3‰ M_{SM}) were added to a reaction flask, heated to 25° C. and reacted for 65 hrs. The reaction solution was quenched with iced water, and alkalized. The organic phase was separated, dried, and concentrated to obtain a crude product of Formula V (4.62 g, purity 90.3%, monofluoro-impurity 0.9%, yield 89.3%).

### Example 4

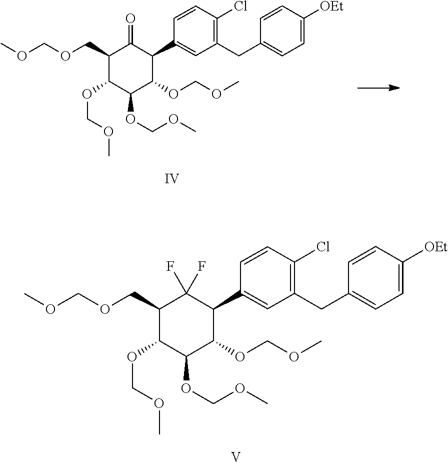

(2R,3R,4R,5S,6S)-3,4,5-tris(methoxymethyloxy)-2-((methoxymethyloxy)methyl)-6-(4-chloro-3-(4-ethoxybenzyl)phenyl)cyclohexanone (5.0 g, 8.4 mmol, 1.0 eq), dichloromethane (16 mL), selective fluorination reagent II (6.8 g, 21.3 mmol, 2.54 eq), and methanol (0.01 g, 2‰ M_{SM}) were added to a reaction flask, heated to 20° C. and reacted for 70 hrs. The reaction solution was quenched with iced water, and alkalized. The organic phase was separated, dried, and concentrated to obtain a crude product of Formula V (4.52 g, purity 90.7%, monofluoro-impurity 2.3%, yield 86.9%).

### Example 5

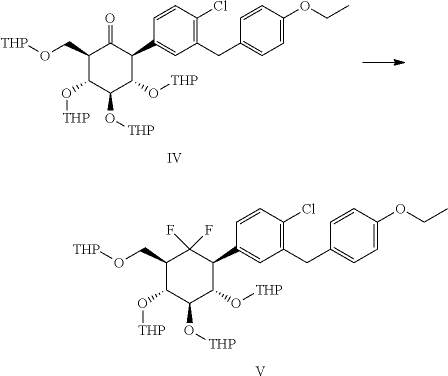

(2R,3R,4R,5S,6S)-3,4,5-tris(2-tetrahydropyranyloxy)-2-((2-tetrahydropyranyloxy)methyl )-6-(4-chloro-3-(4-ethoxybenzyl)phenyl)cyclohexanone (5.0 g, 6.6 mmol, 1.0 eq), tetrahydrofuran (20 mL), bis(2-methoxyethyl)aminosulfur trifluoride (5.2 g, 23.5 mmol, 3.56 eq), and n-propanol (0.01 g, 2‰ M_{SM}) were added to a reaction flask, heated to 19° C. and reacted for 65 hrs. The reaction solution was quenched with iced water, and alkalized. The organic phase was separated, dried, and concentrated to obtain a crude product of Formula V (4.73 g, purity 92.1%, monofluoro-impurity 1.7%, yield 92.0%).

The above embodiments are merely preferred embodiments of the present invention, and the scope of the present invention is not limited thereto. Any insubstantial changes and substitutions made by those skilled in the art based on the present invention fall within the protection scope of the present invention.

What is claimed is:

1. A preparation method for an SGLT2 inhibitor intermediate V, comprising subjecting a compound of Formula IV to a substitution reaction with a fluorination reagent in the presence of a catalytic reagent, 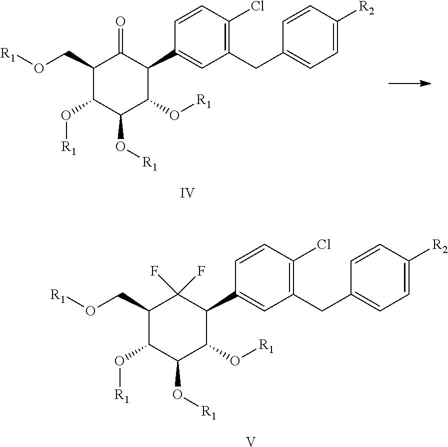
wherein: R₁ is benzyl, p-methoxybenzyl, triphenylmethyl, acetyl, benzoyl, pivaloyl, trimethylsilyl, t-butyldimethylsilyl, t-butyldiphenylsilyl, tri-iso-propylsilyl, 2-tetrahydropyranyl, methoxymethyl or 2-ethoxyethyl, and R₂ is methyl, ethyl, methoxy, ethoxy or (tetrahydrofuran-3-yl)oxy.

2. The preparation method according to claim 1, wherein the compound of Formula IV is dissolved in a solvent, and subjected to a heat-preservation substitution reaction with a fluorination reagent in the presence of a catalytic reagent, to obtain the SGLT2 inhibitor intermediate V.

3. The preparation method according to claim 1, wherein the catalytic reagent is one or more of methanol, anhydrous ethanol, iso-propanol, n-propanol or n-butanol.

4. The preparation method according to claim 1, wherein the amount of the catalytic reagent is 2-6 wt ‰ of the amount of the compound of Formula IV.

5. The preparation method according to claim 2, wherein the solvent is one or more selected from dichloromethane, acetonitrile, isopropyl ether, tetrahydrofuran, methyl tert-butyl ether, n-heptane and ethyl acetate.

6. The preparation method according to claim 2, wherein the amount of the solvent is (0-10) ml/g relative to the weight of the compound of Formula IV.

7. The preparation method according to claim 1, wherein the fluorination reagent is one selected from diethylaminosulfur trifluoride, 1-fluoropyridinium tetrafluoroborate, anhydrous hydrogen fluoride, anhydrous potassium fluoride, DAST fluoroborate, selective fluorination reagent II (CAS No. 159269-48-4,1-fluoro-4-methyl-1,4-diazoniabicyclo[2.2.2]octane;ditetrafluoroborate), bis(2-complex.

8. The preparation method according to claim 1, wherein the molar amount of the fluorination reagent is 2-30 times of the molar amount of the compound of Formula IV.

9. The preparation method according to claim 1, wherein the temperature of the heat-preservation substitution reaction is 10-70° C.

10. The preparation method according to claim 1, wherein the time of the heat-preservation substitution reaction is 10-98 hrs.
